(19) United States
(12) Patent Application Publication
(10) Pub. No.: US 2025/0059105 A1
(43) Pub. Date: Feb. 20, 2025

(54) SOIL ENRICHMENT SYSTEMS AND METHODS

(71) Applicant: MYLAND COMPANY, INC., Phoenix, AZ (US)

(72) Inventors: Andrew D. Ayers, Clay Springs, AZ (US); Mark R. Edwards, Tempe, AZ (US)

(21) Appl. No.: 18/818,095

(22) Filed: Aug. 28, 2024

Related U.S. Application Data
(63) Continuation of application No. 17/524,111, filed on Nov. 11, 2021, now abandoned.
Application No. 17/524,111 is a continuation of application No. 15/647,005, filed on Jul. 11, 2017, now abandoned.
Application No. 15/647,005 is a continuation-in-part of application No. 14/069,932, filed on Nov. 1, 2013, now Pat. No. 10,172,304.
Application No. 14/069,932 is a continuation of application No. PCT/US2012/036293, filed on May 3, 2012.
(60) Provisional application No. 61/481,998, filed on May 3, 2011.

(51) Int. Cl. C05F 11/08; A01G 7/02; A01G 25/16; A01G 33/00; C02F 1/00; C02F 1/28; C02F 1/32; C02F 1/50; C02F 1/68; C02F 3/32; C02F 9/00; C05D 9/02; C05G 5/20; C12M 1/00; C12M 1/12; C12N 1/12

(52) U.S. Cl. CPC C05F 11/08; A01G 7/02; A01G 33/00; C02F 9/00; C05D 9/02; C05G 5/20; C12M 21/02; C12M 23/52; C12M 29/04; C12M 37/00; C12M 43/00; C12N 1/12; A01G 25/16; C02F 1/001; C02F 1/283; C02F 1/325; C02F 1/50; C02F 1/68; C02F 3/322; C02F 2201/782; C02F 2209/008; Y02W 10/37

(57) ABSTRACT

Various embodiments of the present technology provide methods and systems for soil enrichment. The systems may comprise a bioreactor system coupled to an initial treatment system for the cultivation of a live microorganism culture containing organic nutrients on an agriculturally effective scale. The systems may be automated and/or portable for practical applications onto target fields. The live microorganism culture may be delivered onto the soil of the target fields, enriching the soil with the organic nutrients that become bioavailable to crops growing in the soil. The soil enrichment system may provide a sustainable approach to agriculture that may efficiently enhance the natural processes of the native soil of any crop.

### CROSS REFERENCE

This application is a continuation of U.S. patent application Ser. No. 17/524,111, filed on Nov. 11, 2021, entitled “Soil Enrichment Systems and Methods,” which is a continuation of U.S. patent application Ser. No. 15/647,005 filed on Jul. 11, 2017, entitled “Soil Enrichment Systems and Methods,” which is a Continuation-in-Part of U.S. patent application Ser. No. 14/069,932 filed on Nov. 1, 2013, entitled “Microalgae-Based Soil Inoculating System and Methods of Use,” now U.S. Pat. No. 10,172,304, which is a continuation of International Patent Application No. PCT/US2012/036293, filed on May 3, 2012, which claims priority to U.S. Provisional Application Ser. No. 61/481,998, filed May 3, 2011, and incorporates the disclosure of all such applications by reference. To the extent that the present disclosure conflicts with any referenced application, however, the present disclosure is to be given priority.

### BACKGROUND

The cultivation of healthy crops requires an adequate amount of organic matter in the soil. As microorganisms in the soil break down organic matter, a variety of beneficial micro- and macronutrients are released to, and subsequently absorbed by, the plants' roots. This process decreases soil organic matter and affects the soil's ability to sustainably support plant growth.

In natural environments, such as forests or prairies, soil organic matter is replenished as dead plants (e.g., leaves, grass, etc.) fall to the ground, decay, and are tilled into the soil by fauna. The nutrients necessary to grow plants are, in effect, recycled as each generation of plant life dies and decays. However, since crops are continuously harvested, the soil does not receive a steady supply of decaying organic matter needed to naturally replenish the nutrients required for growing crops. Organic matter must be supplemented into the soil for the successful and sustainable crop growth.

The predominant approach for enriching agricultural soil is adding synthetic chemical fertilizers. While synthetic fertilizers may provide nutrients for crop growth, they do not continually replenish the soil organic matter. Some fertilizers, particularly nitrogen, boost microorganism activity in the soil, causing the accelerated consumption of the soil organic matter. Unfortunately, the application of synthetic fertilizers over an extended period of time has contributed greatly to the widespread degradation of the soil in some of the world's most important farming regions.

### SUMMARY

Various embodiments of the present technology provide methods and systems for soil enrichment. The systems may comprise a bioreactor system coupled to an initial treatment system for the cultivation of a live microorganism culture containing organic nutrients on an agriculturally effective scale. The systems may be automated and/or portable for practical applications onto target fields. The live microorganism culture may be delivered onto the soil of the target fields, enriching the soil with the organic nutrients that become bioavailable to crops growing in the soil. The soil enrichment system may provide a sustainable approach to agriculture that may efficiently enhance the natural processes of the native soil of any crop.

### BRIEF DESCRIPTION OF THE FIGURES

A more complete understanding of the present technology may be derived by referring to the detailed description when considered in connection with the following illustrative figures. In the following figures, like reference numbers refer to similar elements and steps throughout the figures.

Elements and steps in the figures are illustrated for simplicity and clarity and have not necessarily been rendered according to any particular sequence or scale. For example, steps that may be performed concurrently or in different order are illustrated in the figures to help to improve understanding of embodiments of the present technology.

The figures described are for illustration purposes only and are not intended to limit the scope of the present disclosure in any way. Various aspects of the present technology may be more fully understood from the detailed description and the accompanying drawing figures, wherein:

FIG. 1 representatively illustrates an exemplary embodiment of the soil enrichment system;

FIG. 2 is a block diagram of an exemplary remotely controlled soil enrichment system;

FIG. 3 is a flow chart of an exemplary method of using the soil enrichment system to enrich the soil of a target field;

FIG. 4 representatively illustrates an exemplary embodiment of the soil enrichment system contained inside a portable housing; and

FIG. 5 is a block diagram of a soil enrichment system according to various aspects of the present technology.

### DETAILED DESCRIPTION OF EXEMPLARY EMBODIMENTS

The present technology may be described in terms of functional block components and various processing steps. Such functional blocks may be realized by any number of components configured to perform the specified functions and achieve the various results. For example, the present technology may employ various process steps, apparatus, systems, methods, materials, etc., for filtering, pumping, flow control, fluid storage and transfer, and mixing. In addition, the present technology may be practiced in conjunction with any number of devices used to culture microorganisms, provide nutrient solutions, monitor growth characteristics of the culture, and deliver the culture to an agricultural crop, and the systems described are merely exemplary applications for the technology.

The particular implementations shown and described are illustrative of the technology and its best mode and are not intended to otherwise limit the scope of the present technology in any way. For the sake of brevity, conventional manufacturing, preparation, process steps, and other functional aspects of the system may not be described in detail. Furthermore, connecting lines shown in various figures are intended to represent exemplary functional relationships and/or steps between the various elements. Many alternative or additional functional relationships or process steps may be present in practical systems and methods.

Various embodiments of the present technology provide a soil enrichment system for culturing and delivering live microbial organisms, such as live microalgae, onto soil. Live microalgae may provide nutrients for assimilation by crops grown in the soil, such as the fixation of atmospheric nitrogen by blue-green algae, which makes nitrogen available to the crops. Beneficial organic compounds may be released by the microalgae while they are alive and as they die and decay. The particular profile of nutrients provided by the microalgae may depend upon the strain or species of microalgae used in the soil enrichment system.

In various embodiments, use of the soil enrichment system may improve overall crop yield by approximately 5% to 39% or higher, as compared to untreated crops. In some embodiments, soil enriched with microalgae from the soil enrichment system may produce crops exhibiting improvements in the texture, taste, size, nutrient content, and/or yield of a crop as compared to an untreated crop.

Application of the soil enrichment system to soil may result in reductions in one or more of total energy consumption, ecological pollution, greenhouse gas emission, use of chemical fertilizers, overall crop production cost, tillage cost, need for and use of fungicides, herbicides and/or pesticides, soil compaction, consumption of irrigation water, occurrence of overfertilization, and/or run-off and soil erosion, as compared to untreated crops. Similarly, use of the soil enrichment system may result in increases in bioavailability of micronutrients and macronutrients to the crop, soil porosity, microbial activity within soil, water/moisture retention by soil, and/or organic content of soil, as well as improvements in desirable plant characteristics, as compared to untreated crops.

Various embodiments of the soil enrichment system may be applied to any soil used for the growth of any plants, regardless of whether the plants are grown for aesthetic reasons or for consumption. For example, the soil enrichment system may be applied to any soil-based farm, parks, hydroponic farms, aquaponics, nurseries, golf-courses, sporting fields, orchards, gardens, zoos, and any other places where crops or plants are grown.

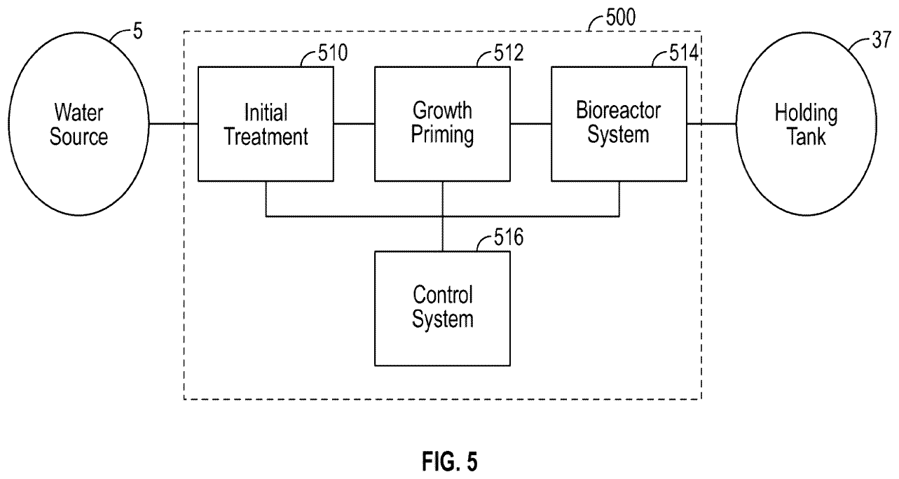

Referring to FIG. 5, methods and apparatus for soil enrichment 500 according to various aspects of the present invention may operate in conjunction with an initial treatment system 510, a growth priming system 512, and a bioreactor system 514. The initial treatment system 510 may treat incoming water to prepare the water for processing by the growth priming system 512 and the bioreactor system 514, for example by filtering and/or sterilizing the water. The growth priming system 512 adds material to grow the live microbial organisms, such as nutrients, to the water. The bioreactor system 514 provides an environment for the live microbial organisms to grow prior to dispensation to a target area. The soil enrichment system 500 may further comprise a control system 516 for monitoring the status and controlling operation of various elements of the soil enrichment system 500.

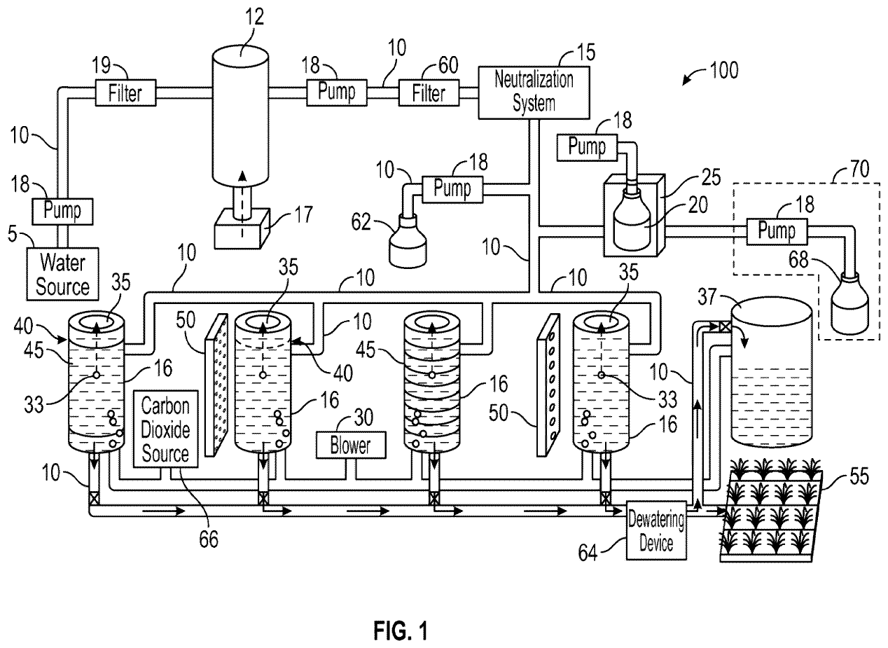

Various embodiments of the soil enrichment system 500 may comprise one or more components and may comprise more than one of any individual component. For example, referring to FIG. 1, an exemplary soil enrichment system 100 implementation may include an exemplary initial treatment system 510 comprising a solids filter 19, a water storage tank 12, a sterilization system 17, and a neutralization system 15. An exemplary growth priming system 512 may comprise one or more nutrient solution feeds, such as first and second nutrient solution containers 20, 62 to add nutrient solutions to the treated water. An exemplary bioreactor system 514 may comprise one or more bioreactors 16 to facilitate inoculation with and growth of the microorganism. The systems and methods may include various additional systems and subsystems, such as one or more nutrient solution containers, refrigerators, light sources, blowers, carbon dioxide sources, pumps, valves, fluid conduits, air conduits, gas conduits, air filters, gas filters, control systems, sensors, air conditioning units, exhaust systems, portable housings, and/or exterior holding tanks.

Various components may comprise an inlet and/or an outlet for transporting fluid in and out. The inlets and/or outlets may be coupled to one or more fluid conduits to provide fluid communication into and out of each component. In various embodiments, the inlet and/or outlet of each component may be coupled to its fluid conduit through any suitable fitting for joining and adapting the fluid conduit. For example, the fitting may comprise an appropriate sealant such as PVC cement or other adhesives and/or sealants, compression fittings, insert fittings, combination fittings, friction fittings, and/or adapters.

The inlet and the outlet may be configured to be coupled to the fluid conduits such that irrigation water and other fluids may travel through the soil enrichment system 500. The arrangement of components within an exemplary soil enrichment system 500 may provide for a substantially automated flow-through system for growing one or more desired strains of microorganisms, for example on an agricultural scale, for soil enrichment.

Fluid conduits may comprise any suitable hollow tubing and/or pipe appropriate for transportation of the relevant fluids. For example, the fluid conduits may comprise PVC pipe, CPVC pipe, metal pipe, flexible plastic hose, and/or flexible rubber hose. In some embodiments, the fluid conduits may be the same throughout the soil enrichment system, or the fluid conduits may be varied to accommodate various specifications, such as transparency for monitoring and control purposes (e.g., camera imaging) or to meet requirements for the internal diameter of the fluid conduit to move fluid slowly, quickly, and/or according to a desired pressure.

The soil enrichment system 500 and its various components may be portable, for example to allow the soil enrichment system to operate in a non-permanent location. For example, in some embodiments, all or portions of the soil enrichment system may be coupled to and/or disposed within a portable housing (i.e., the housing may be capable of being moved). Portable embodiments of the soil enrichment system 500 may be configured for remote operation, substantially continuous production of one or more live microorganism cultures, and/or delivery of live microorganism cultures directly onto the target field.

The portable housing may be configured to contain and support the soil enrichment system 500. For example, the portable housing may comprise a wheeled trailer that may be towed by a vehicle from one site on the target field to another site on the same target field or a different target field altogether. In another example, the portable housing may comprise a conventional shipping container, such as a steel shipping container configured to be lifted onto a flatbed truck, trailer, and/or train by a fork lift or crane. The conventional shipping container may be any suitable size and/or dimensions to accommodate the size of the soil enrichment system 500. The soil enrichment system 500 may be configured to operate entirely or partially inside the portable housing.

The components of the soil enrichment system 500 may be secured to the portable housing, such as via the interior walls, floor, and/or ceiling. The components may be secured with any suitable fasteners, such as clamps for securing the fluid conduits to the walls of the portable housing, shelving bolted to the walls and/or the ceiling to which various components such as the sterilization system may be secured, and one or more frames for securing one or more bioreactors to the floor. The fasteners may prevent relative rotation and/or longitudinal movement of components during transport and operation of the soil enrichment system to ensure their integrity and stability.

The soil enrichment system 500 may be configured to allow flexibility in the location of operation of the soil enrichment system and in the method of delivery of the microorganism culture to the receiving location. For example, the soil enrichment system 500 may be operated remotely from the target field, such as in a warehouse, parking lot, barn, etc., where the microorganism culture may flow through the outlet and into an external holding tank. The external holding tank may then be transported to the target field for delivery of the microorganism culture. In another example, the soil enrichment system 500 may be operated remotely and then towed to a target field to continue to operate and/or to allow the microorganism culture to flow through the outlet and onto the target field.

In various embodiments, the microorganism culture produced by the soil enrichment system 500 may be directed to any suitable receiving location. The receiving location may comprise the target field, an exterior holding tank, and/or any suitable microorganism culture harvesting and/or storage apparatus. In some embodiments, the soil enrichment system 500 may operate in one location and later be transported to the receiving location. In other embodiments, the soil enrichment system 500 may operate in one location and the microorganism culture may be harvested and transported to the receiving location. For example, where the target field is located remotely from an external water source to which the soil enrichment system is coupled, the microorganism culture may flow through an outlet into an exterior holding tank located in the portable housing and/or exterior to the portable surface. The exterior holding tank may then be transported to the target field for delivery of the microorganism culture or for further dewatering and/or storage of the microorganism culture. In another example, where the target field is located proximate to the external water source, the microorganism culture may flow through the outlet directly onto the target field.

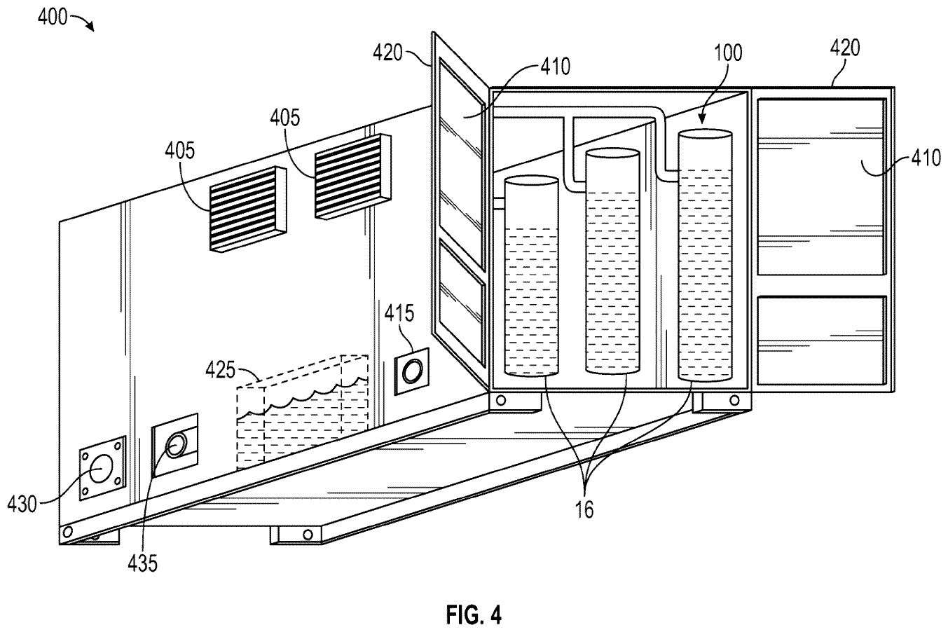

For example, referring to FIG. 4, in some embodiments, the soil enrichment system 500 may be inside and coupled to a portable housing 400, such as a conventional shipping container. The portability of the soil enrichment system 500 may allow for transport of the soil enrichment system from location-to-location. Any one or more of the components within the soil enrichment system 500 may be coupled to the portable housing 400.

The portable housing 400 may be accessible through doors 420 such that people and components of the soil enrichment system 500 may enter. In some embodiments, the doors 420 may be equipped with airtight panels 410 configured to provide an airtight seal when the doors 420 are closed. The airtight seal may inhibit contamination of the bioreactors 16. The portable housing 400 may comprise environmental controls, such as heating ventilation, air conditioning, and humidity control systems 405 to regulate the ambient environment within the portable housing 400.

In some embodiments, the portable housing 400 may comprise an access port 415 for coupling or feeding through a fluid conduit connected to the water source 5 located externally to the portable housing 400. In some embodiments, the portable housing 400 may contain a water reservoir 425 configured to be the water source 5 for supplying irrigation water. In some embodiments, the portable housing 400 may comprise inlet 430 for receiving a gas conduit, such as from the carbon dioxide source. In some embodiments, the portable housing 400 may comprise a port 435 configured to provide an opening for a fluid conduit carrying the microorganism culture for delivery to the target field 55 and/or the external holding tank 37.

Referring again to FIG. 5, a water source 5 supplies water to the soil enrichment system 500. Water flowing from the water source 5 may be referred to as “irrigation water.” The water source 5 may comprise any suitable source of irrigation water appropriate for irrigation of plants. In some embodiments, the water source 5 may be under pressure, such as water from a well or a public utility in a city, town, or municipality. In some embodiments, the water source 5 may be substantially unpressurized. For example, the water source 5 may comprise a stationary water reservoir, reclaimed wastewater, well water, lake water, creek water, pond water, rainwater, river water, and/or freshwater.

In various embodiments, irrigation water may be supplied to the soil enrichment system through an irrigation pipe in the ground and/or through irrigation water stored in an irrigation water storage tank. The irrigation water storage tank may be configured to temporarily hold the irrigation water and transfer water to the soil enrichment system 500 in any appropriate manner. For example, referring to FIG. 1, when a substantially unpressurized water source is used, a pump 18 may be coupled to the water source 5 to pump the irrigation water under pressure into the soil enrichment system 500. In some embodiments, the irrigation water chosen for use in the soil enrichment system 500 may exhibit low salinity and/or be free from heavy metals to support the growth of the microbes and/or the plants to be grown in the soil enriched with the microbes.

In one embodiment, the irrigation water storage tank may be placed on the portable surface or inside the portable housing, such that the soil enrichment system is completely contained and capable of operation in the portable housing. In other embodiments, the soil enrichment system may be transported in the portable housing to a location proximate to an outlet for the water source 5 and may remain in that location for operation.

The initial treatment system 510 treats incoming water to prepare the water for processing by the growth priming system 512 and the bioreactor system 514. The initial treatment system 510 may treat the water according to any appropriate criteria, for example filtering and/or sterilizing the water to remove materials that may harm the soil enrichment system 500 and/or inhibit growth of the organisms to be grown in the water. In one embodiment, the initial treatment system 510 filters contaminants from the water and sterilizes the water to remove all living organisms from the water. Referring again to FIG. 1, an exemplary initial treatment system 510 may comprise one or more filters 18, 19, a sterilization system 17, and a neutralization system 15.

The filter may remove solids and/or other undesirable materials from the irrigation water, such as before the irrigation water enters the bioreactor system 514. In some embodiments, the first component of the soil enrichment system that the irrigation water passes through may be a first solids filter 19. The first solids filter 19 may be configured to remove solids, such as solids greater than a particular size, from the irrigation water to form a filtered irrigation water. In some embodiments, a second solids filter 60 may be configured to remove solids from sterilized irrigation water flowing from the water storage tank 12 to the neutralization system 15.

In various embodiments, the first solids filter 19 and the second solids filter 60 may comprise a variety of suitable filters configured to remove solids. In various embodiments, the first solids filter 19 and the second solids filter 60 may comprise: media filters, disk filters, screen filters, microporous ceramic filters, carbon-block filters, carbon-resin filters, granulated carbon, carbon impregnated filter media, membrane filters, microporous media filters, reverse osmosis filters, slow-sand filter beds, rapid-sand filter beds, and/or cloth filters. In some embodiments, the first solids filter and/or the second solids filter may comprise flow-through filters. For example, the solids filter may comprise a polypropylene microfiber pleated bag filter, such as an X100 pleated bag filter available from www.filterbag.com, or a polypropylene filter vessel with a reusable bag filter, such as an FV1 bag filter available from www.pentairaes.com. In some embodiments, the first solids filter and second solids filter may be the same type of filter. In other embodiments, the first solids filter may be a different type of filter from the seconds solids filter.

A water storage tank 12 may store the irrigation water or the filtered irrigation water. The water storage tank 12 may comprise any suitable container, and may be selected according to size, form factor, materials, or other suitable criteria. The water storage tank 12 may also provide a container for treatment of the water, such as sterilization.

In one embodiment, the water storage tank 12 may comprise an opening capped with a valve. A fluid conduit may be attached to the valve for transferring water from the water source 5 and/or the first solids filter 19, for example to provide a path for the irrigation water and/or the filtered irrigation water to enter the water storage tank 12. The water storage tank 12 may comprise any suitable container for holding water, such as flexible bladders, steel tanks, epoxy lined steel tanks, glass tanks, and/or polyethylene tanks. For example, the water storage tank may comprise a high or low density polyethylene material.

In some embodiments, the water storage tank 12 may be elevated above the other components of the soil enrichment system 500, such as to gravity feed the irrigation water and/or the filtered irrigation water to an input pump. In some embodiments, the water storage tank 12 may have a cone-shaped bottom. In some embodiments, the water storage tank 12 may comprise an opaque, black, and/or other light blocking material to reduce or eliminate the exposure of the irrigation water and/or the filtered irrigation water in the water storage tank 12 to light to impede the growth of unwanted microbes.

Various embodiments of the initial treatment system 510 may comprise a sterilization system 17 configured to substantially sterilize the irrigation water and/or filtered irrigation water while in the water storage tank 12. Treatment of the irrigation water and/or filtered irrigation water with the sterilization system 17 may produce sterilized irrigation water. In various embodiments, the sterilization system 510 may comprise any suitable chemical and/or apparatus that effects sterilization of the irrigation water and/or filtered irrigation water, such as an ozone generator, a chlorine generator, a heat source for boiling, pressurized steam, radiation, and a dissolved oxygen generator. For example, in some embodiments, the water storage tank 12 may comprise an outlet coupled to a valve for connection to a fluid conduit, such as a hose, to the ozone generator or the chlorine generator, which may inject ozone gas or chlorine, respectively, into the water storage tank containing the irrigation water and/or filtered irrigation water. The irrigation water and/or filtered irrigation water may remain in the water storage tank 12 for a pre-selected residence time to allow the sterilization process to kill all organisms in the water.

Various embodiments of the sterilization system 17 may comprise an ozone generator. In some embodiments, the ozone generator may be configured to generate ozone gas, O₃, from dry air. In some embodiments, the ozone generator may be coupled to a dissolved oxygen generator configured to generate 90% oxygen. The ozone gas produced by the ozone generator may kill unwanted microbes present in the irrigation water and/or the filtered irrigation water. The ozone gas may also degrade organic contaminants such as herbicides, pesticides, and fungicides that may harm microorganisms cultured in the bioreactor system if they are not degraded. The ozone generator may comprise any suitable system for producing ozone gas, such as model O1 by Pacific Ozone, the Nano by Absolute Ozone, the OZ8PC20 by Ozotech, or the AirSep Topaz series.

In various embodiments, the residence time of the irrigation water and/or the filtered irrigation water in the water storage tank 12 for sterilization may correlate to one or more of their quantity, the amount of microbes present, and/or the amount of organic contaminants present. For example, the ozone gas may be present in the irrigation water and/or the filtered irrigation water in the water storage tank 12 for approximately 24 hours prior to allowing the sterilized irrigation water to exit the water storage tank 12 and proceed to the inlet pump. The concentration of ozone gas applied to the irrigation water and/or the filtered irrigation water in the water storage tank 12 may be approximately 0.2 parts per million (ppm) to approximately 0.5 ppm of ozone gas. Sterilization may be further improved by employing a gas diffuser in the water storage tank 12, wherein the gas diffuser is configured to more effectively distribute the ozone gas to the irrigation water and/or the filtered irrigation water.

Various embodiments of the sterilization system 17 may comprise a chlorine generator. In some embodiments, the chlorine generator may comprise a chlorine generator cell that produces hypochlorous acid from salt. In some embodiments, the dose of hypochlorous acid to be injected into the water storage tank 12 may be controlled by a dosing pump and a control system, such as the PLC system described below.

Various embodiments of the soil enrichment system 500 may comprise an input pump 18, such as a conventional water pump. In some embodiments, the sterilized irrigation water may flow out of the water storage tank 12 through a fluid conduit, wherein the fluid conduit may be coupled to the an inlet of the input pump 18. Another fluid conduit may be coupled to an outlet of the input pump 18, wherein the sterilized irrigation water may be propelled out of the outlet of the input pump 18 at a faster speed and/or pressure than the sterilized irrigation water entered through the inlet of the input pump 18.

The input pump 18 may be switched on to propel sterilized irrigation water through the neutralization system 15 and neutralized irrigation water into the bioreactor system 514. The input pump 18 may be switched off to stop the flow of sterilized irrigation water and neutralized irrigation water. Control of the input pump 18 may maintain adequate water levels in the bioreactor system 514. In some embodiments, a flow switch may be coupled to the outlet of the input pump 18 and configured to regulate flow rate and/or pressure through the input pump 18.

Various embodiments of the soil enrichment system 500 may comprise the neutralization system 15 for removing or neutralizing any chemicals used to effect sterilization to produce the sterilized irrigation water. Neutralization of these chemicals may prevent the chemicals from killing and/or impeding the growth of microbes grown in the bioreactor system 514. Various embodiments of the neutralization system 15 may be configured as a flow-through system in which the chemicals in the sterilized irrigation water are neutralized as the sterilized irrigation water passes through the neutralization system 15.

The sterilized irrigation water may enter into, pass through, and/or exit from the neutralization system 15 through a fluid conduit. For example, in one embodiment, the sterilized irrigation water may exit the water storage tank 12 through the fluid conduit and enter the second solids filter 60 and then exit the second solids filter 60 through a fluid conduit and enter the neutralization system 15. The second solids filter 60 may comprise a portion of the neutralization system 15, the sterilization system 17, or the water storage tank 12, or it may be a separate component. In another embodiment, the second solids filter 60 is omitted, and the sterilized irrigation water may exit the water storage tank 12 through a fluid conduit to enter the neutralization system 15.

The sterilized irrigation water may be treated by the neutralization system 15 to form neutralized irrigation water. In some embodiments where the sterilization system 17 comprises an ozone generator, treatment of the sterilized irrigation water with the neutralization system 15 may form deozonated irrigation water due to the removal and/or degradation of the ozone. In some embodiments where the sterilization system 17 comprises a chlorination system, treatment of the sterilized irrigation water with the neutralization system 15 may form dechlorinated irrigation water due to the removal and/or degradation of the chlorine.

In various embodiments, the neutralization system 15 may comprise an ultraviolet (UV) light system, a carbon filter, or a combination of both. A UV light system or a carbon filter may be used to neutralize ozone. A combination of a UV light system and carbon filter may be used to neutralize chlorine. In some embodiments, the neutralization system 15 may comprise a conventional dechlorination system to neutralize chlorine. Conventional dichlorination systems may typically add sulfur dioxide, sulfite salts, or hydrogen peroxide to remove residual chlorine.

The UV light system may expose the sterilized irrigation water to UV radiation to degrade ozone (deozonation) or chlorine (dechlorination), producing neutralized irrigation water. In some embodiments, the UV light system may be configured as a flow-through system in which the sterilized irrigation water is deozonated and/or dechlorinated as it passes through the UV light system. Suitable UV systems may include the CSL Series by Aquafine, and the UVS3XX Series by UV Sciences (www.aquafineuv.com; Valencia, CA). In some embodiments, the UV light system may further sterilize the sterilized irrigation water as UV radiation itself kills microbes and degrades organic compounds.

The carbon filter may be configured as a flow-through system in which the sterilized irrigation water is deozonated or dechlorinated as it passes through the carbon filter, producing neutralized irrigation water. The carbon filter may generally employ activated carbon, such as granule and/or powdered activated carbon. In some embodiments, the carbon filter may comprise a minimum of approximately 0.65 kg of activated carbon. Suitable carbon filters may include, for example, the 20″ Carbon Block Cartridge Filter System from Filter Water (www.filterwater.com).

In various embodiments of the soil enrichment system 500, the neutralization system 15 may be coupled to the bioreactor system 514 through the growth priming system 512. The growth priming system 512 may add a nutrient solution to the water including one or more crop nutrients, such as macronutrients, micronutrients, and/or nutritional media such as conventional microalgae feed or bacterial growth media.

For example, referring again to FIG. 1, in some embodiments, one or more nutrient solution containers 20, 62 may be coupled to the fluid conduit positioned between the neutralization system 15 and the bioreactor system 514. Each of the nutrient solution containers 20, 62 may contain a nutrient solution for delivery into the bioreactor system 514. The nutrient solution may comprise a vitamin solution. Another nutrient solution may comprise a mineral solution. In some embodiments, one or more nutrient solution containers 20, 62 may be positioned downstream of the neutralization system 15 to prevent the degradation of the nutrient solution and/or to prevent the nutrient solutions from impacting the effectiveness of the neutralization system 15.

The solution containers 20, 62 may be housed in a refrigerator if a temperature lower than the ambient temperature in the portable housing is needed to maintain the nutrient solution. For example, the vitamin solution may need refrigeration at 4° C. while the mineral solution may be stored at the ambient temperature.

In some embodiments, the nutrient solution containers 20, 62 may comprise any container configured to be sterilized and maintain the sterility of its internal volume. The one or more nutrient solution containers 20, 62 may hold a volume of nutrient solution and provide an outlet to allow the nutrient solution to be added to the neutralized irrigation water. In some embodiments, the one or more nutrient solution containers 20, 62 may comprise a material that may be autoclaved, such as polypropylene, glass, and/or a fluoropolymer. The one or more nutrient solution containers 20, 62 may comprise a sterile vent and/or valve to maintain the sterility of the nutrient solution. In various embodiments, a pump 18 may be coupled to the one or more nutrient solution containers 20, 62 to provide a pre-selected dose of the nutrient solution into the neutralized irrigation water. In some embodiments, the fluid conduit may comprise one or more valves that may control the delivery of neutralized irrigation water and/or nutrient solutions to the one or more bioreactors 16.

Referring again to FIG. 5, various embodiments of the soil enrichment system 500 may comprise the bioreactor system 514. The bioreactor system 514 may comprise one or more of any suitable bioreactor comprising a microorganism growth chamber configured to receive a microorganism inoculant and/or provide an internal environment that supports the growth of the microorganism inoculant. For example, referring again to FIG. 1, the bioreactor system 514 may include a 500 gallon cylindrical stirred tank vessel configured for maintaining the sterility of its internal volume and growing the microorganism inoculant as a suspension culture. In various embodiments, the bioreactor system 514 may include a batch bioreactor or a continuous bioreactor. The neutralized irrigation water and nutrient solution may enter the one or more bioreactors 16 through a valve coupled to the fluid conduit.

The bioreactors 16 may be cleaned by introducing a cleaning solution into the bioreactors, such as through the valve and/or fluid conduits. The cleaning solution may comprise any suitable chemical rinse for sterilizing the interior of the one or more bioreactors 16, such as a dilute bleach solution or sulfuric acid solution. The cleaning solution may remain in the one or more bioreactors 16 for a sufficient time to kill any microorganisms, such as bacteria and/or algae, that may be in the one or more bioreactors 16. The cleaning solution may then be removed from the one or more bioreactors 16, such as by suction and/or through an outlet coupled to the one or more bioreactors 16. In some embodiments, the interior walls of the one or more bioreactors 16 may be manually scrubbed. In some embodiments, the bioreactors 16 may be sterilized with the cleaning solution prior to inoculation with the microorganism inoculant. For example, the one or more bioreactors 16 may be sterilized with the cleaning solution for approximately an hour prior to inoculation.

Some embodiments of the bioreactor system 514 may comprise an automated cleaning system 70 controlled by the control system 516. The automated cleaning system 70 may comprise a cleaning solution container 68 for holding the cleaning solution and a pump 18 for pumping the cleaning solution from the cleaning solution container 68 into the fluid conduit and/or the one or more bioreactors 16. In some embodiments, each of the one or more bioreactors 16 may comprise a dedicated valve for connection of a fluid conduit leading to the cleaning solution container 68 for the cleaning solution.

In various embodiments, each of the one or more bioreactors 16 in the bioreactor system 514 may be the same or different from one another in capacity and/or type of bioreactor. Each of the bioreactors 16 may comprise any suitable material such as glass and/or plastic. In some embodiments, the material may be substantially transparent to allow light to penetrate into the bioreactors 16 for supporting the growth of a microorganism inoculant comprising photosynthetic microorganisms, such as algae and/or photosynthetic bacteria, to grow into a microorganism culture.

In various embodiments, each bioreactor 16 in the bioreactor system 514 may comprise any number of ports that provide access to an interior volume of the bioreactor 16. For example, in one embodiment, a port may be coupled to the fluid conduit. In another embodiment, a port may provide various sensors access to the microorganism culture.

In some embodiments, the bioreactor system 514 comprising multiple bioreactors 16 may be configured to grow a plurality of different microorganism inoculants. For example, each of the bioreactors 16 may grow: a) the same microorganism inoculant, such as an axenic culture of microalgae, or b) two or more different types of microorganism inoculants, such as two or more different strains of microalgae. In some embodiments, each of the bioreactors 16 may contain a different microorganism inoculant. For example, one bioreactor 16 may contain a strain of microalgae and another bioreactor 16 may contain two different strains of bacteria and yet another bioreactor 16 may contain a mixture of two different strains of microalgae. The selection of microorganism inoculant may be based on the nutritional needs of the crop or plants onto which the resulting microorganism culture may be released.

The volume and/or flow rate of neutralized irrigation water and nutrient solution may be regulated to provide the appropriate level of microorganism culture penetration into the soil. For example, a 200-acre target field may receive a total daily volume of 100 to 200 gallons of microorganism culture at a delivery rate of 4.17 to 8.33 gallons/hour that may be diluted with additional irrigation water and/or neutralized irrigation water prior to application to the soil.

In an exemplary embodiment, the microorganism culture may comprise a cell titer (the cell count) in each of the one or more bioreactors 16 which may fluctuate over time. The cell titer may comprise a metric that relates to the microorganism culture's health and productivity. The cell titer along with the microorganism cell weight may be used to measure the biomass delivered to the soil. The cell titer and cell weight may be strain and/or species specific and various metrics may need to be measured to determine each species viability as a microorganism inoculant. For example, the microorganism culture may comprise microalgae as the microorganism. The microalgae cell titer may comprise at least approximately 1,000,000 cells per milliliter of microorganism culture up to approximately 25,000,000 cells per milliliter of microorganism culture. The cell titer may also be strain specific, and can be higher or lower than the range stated above.

Various embodiments of the bioreactor system 514 may comprise a carbon dioxide source 66 to supply a carbon source to the microorganism culture. Carbon dioxide may be added directly and/or indirectly to the one or more bioreactors. The carbon dioxide source 66 may be a tank containing carbon dioxide gas, a carbon dioxide generator, a carbon dioxide-sequester for sequestering and temporarily storing atmospheric carbon dioxideor a combination thereof. In some embodiments, carbon dioxide captured from air may be used, such as using methods disclosed in Method and Apparatus for Extracting Carbon Dioxide from Air, U.S. Pat. No. 8,083,836 (filed Oct. 13, 2010). The carbon dioxide source 66 may be coupled to the one or more bioreactors through a gas conduit coupled to the port. The one or more bioreactors may comprise a valve for regulating the entry of carbon dioxide from the carbon dioxide source 66.

Atmospheric air contains approximately 0.035-0.04% wt. of carbon dioxide. While atmospheric air can serve as a source of carbon dioxide for the microorganism culture, the concentration of carbon dioxide is generally too low to sustain the rapid proliferation of microorganisms in the one or more bioreactors. Accordingly, carbon dioxide may be added to air that may be injected into the one or more bioreactors through a blower, as described below. The concentration of carbon dioxide in the air added to the one or more bioreactors may be in the range of approximately 1-3% wt., 1.5-2.5% wt., 1.8-2.2% wt., or about 2% wt. In some embodiments, the carbon dioxide may be added directly to the one or more bioreactors and the volume of carbon dioxide may be controlled by a pH controller. When the pH of the microorganism culture rises above pH 8.0, the control system 516 may open a valve to allow carbon dioxide to flow into the one or more bioreactors. The carbon dioxide may dissolve into the water producing carbonic acid which lowers the pH of the microorganism culture.

Various embodiments of the bioreactor system 514 may include systems to enhance the growth of the microorganisms, such as mixing systems and/or aerating systems. For example, the bioreactor system 514 may include an aerating system, such as air pump or blower 30, coupled to the bioreactor 16 through an air conduit and configured to aerate a microorganism culture in the bioreactor 16. In some embodiments, the blower 30 may be configured to inject sterile air into the one or more bioreactors 16 through a valve configured to regulate the entry of sterile air into the one or more bioreactors 16. The valve may be controlled by the control system 516. For example, air from the blower 30 may be filtered to sterilize the air before it enters the one or more bioreactors 16. In some embodiments, air from the blower 30 may be combined with carbon dioxide from the carbon dioxide source and be delivered into the one or more bioreactors 16 through the air conduit.

In some embodiments, the blower 30 may be coupled to other portions of the soil enrichment system 500 to create positive pressure. The positive pressure throughout the soil enrichment system 500 may reduce or prevent contamination of the components from airborne dust, microorganisms, and/or moisture. For example, the blower 30 may also provide a flow of air to the water storage tank 12 and/or an external holding tank. In some embodiments, air from the blower 30 may be combined with ozone prior to delivery of the air to the water storage tank 12.

The bioreactor system may further include or cooperate with environmental control systems. For example, various embodiments of the soil enrichment system 500 may comprise one or more heaters to heat irrigation water, sterilized irrigation water, and/or neutralized irrigation water as they are conducted through the fluid conduits. In some embodiments the heater may heat the microorganism culture in the bioreactor 16, for example to maintain an optimal temperature of the microorganism culture during cold weather.

In some embodiments, the soil enrichment system 500 may comprise an air conditioning system to regulate the temperature of ambient air within the portable housing. The ambient air may need to be cooled during hot summer months or heated during cold weather to maintain the integrity of various materials and electronics used in the components of the soil enrichment system 500.

In various embodiments, the one or more bioreactors 16 may be used to grow a microorganism culture comprising a photosynthetic microorganism. To grow the photosynthetic microorganism, the one or more bioreactors 16 may be configured to allow the light source to penetrate into the microorganism culture. In some embodiments, the light source may comprise natural sunlight. The natural sunlight may be reflected and/or bent to reach the one or more bioreactors 16. In one embodiment, the bioreactor 16 may comprise a wall that may be at least partially transparent to light to allow natural light to penetrate into the microorganism culture.

In another embodiment, the light source may comprise an artificial light source. The artificial light source may comprise any suitable light source configured to provide adequate light in intensity and wavelength to grow the photosynthetic microorganism. For example, in some embodiments, the artificial light source may comprise a plurality of light-emitting diodes (LEDs), such as a LED tubes and/or sheets. In another embodiment, the light source may comprise conventional light bulbs, fluorescent light tubes, fiber optic light, and the like. The artificial light source may be disposed inside the one or more bioreactors 16, embedded into the wall of the one or more bioreactors 16, and/or coupled to the one or more bioreactors 16. In one embodiment, the artificial light source may be submerged in the microorganism culture as it grows within the bioreactor 16, such as is described in Algae Cultivation Systems and Methods, U.S. Pat. No. 8,033,047 (filed Oct. 23, 2008).

In some embodiments, a fluid conduit coupled to the outlet of the one or more bioreactors 16 and/or coupled to an inlet of the exterior holding tank may also use natural and/or artificial light. For example, the fluid conduit may include a light source to illuminate the contents of the conduit. In another embodiment, the fluid conduit may be exposed to natural sunlight including reflected and/or bent sunlight.

Various embodiments of the soil enrichment system 500 may comprise a dewatering device 64 for harvesting the microorganism culture. The dewatering device 64 may concentrate the microorganism culture into a concentrated microorganism slurry of any desired density. The dewatering device 64 may be coupled to the bioreactor system 514, such as through one or more fluid conduits connected to an outlet of the one or more bioreactors 16 and an inlet of the dewatering device 64. In some embodiments, the soil enrichment system 500 may comprise more than one dewatering device 64. The dewatering device 64 may be configured to deliver the concentrated microorganism slurry to the target field 55 and/or the exterior holding tank 37. The dewatering device 64 may concentrate the microorganism culture through any suitable process such as, but not limited to: 1) flocculation and sedimentation; 2) flotation and collection; and/or 3) centrifugation.

In some embodiments, the dewatering device 64 may be configured to produce the concentrated microorganism slurry through the flocculation and sedimentation process by adding a compound to the microorganism culture which causes the microorganism cells to clump together and fall to the bottom of the dewatering device's 64 tank. The clarified water may be removed from the top of the dewatering device's 64 tank, leaving the concentrated microorganism slurry at the bottom of the dewatering device's 64 tank. The concentrated microorganism slurry may then be pumped into the target field and/or the exterior holding tank.

In some embodiments, the dewatering device 64 may be configured to produce the concentrated microorganism slurry through flotation and collection by adding a compound to the microorganism culture which causes each microorganism cell to have a slight electrical charge. Subsequently, the dewatering device 64 may add microbubbles into the microorganism culture. The electrically charged microorganism cells may be attracted to the bubbles and floated to the surface of the microorganism culture, where they are skimmed off the surface and pumped into the target field and/or the exterior holding tank.

In some embodiments, the dewatering device 64 may be configured to produce the concentrated microorganism slurry through the process of centrifugation. In various embodiments, the microorganism culture may be centrifuged using a solid bowl centrifuge and/or a disk stack centrifuge. The solid bowl centrifuge may collect the microorganisms in the microorganism culture in the inner surface of a rotating bowl. The resulting microorganism slurry may be thick and may be scrapped from the rotating bowl and delivered onto the target field and/or the exterior holding tank. The disk stack centrifuge may comprise a large stack of rotating stainless steel funnel-shaped pieces. Microorganisms may collect on the surface of the funnel-shaped pieces and may flow to a harvest basin where the microorganism slurry collects until it is removed. The speed of the rotating discs may determine the density of the concentrated microorganism slurry produced. The discharged concentrated microorganism slurry may be delivered onto the target field and/or the exterior holding tank.

Various embodiments of the soil enrichment system may comprise an external holding tank configured to receive the microorganism culture. Some embodiments of the soil enrichment system may comprise more than one external holding tank. The external holding tank may be configured to hold the microorganism culture and/or the concentrated microorganism slurry and may maintain their sterility.

In some embodiments, the external holding tank may be configured to support continued growth of the microorganisms in the microorganism culture and/or the concentrated microorganism slurry. For example, in some embodiments, the neutralized irrigation water and/or the nutrient solution may be delivered into the external holding tank to support continued growth and/or health of a photosynthetic microorganism until it is delivered onto the target field.

Referring again to FIG. 1, in various embodiments, the microorganism culture may be released from the one or more bioreactors 16 through the outlets, flow through the one or more fluid conduits, and flow into the external holding tank 37 for storage. In various embodiments, the external holding tank 37 may comprise an at least partially transparent material such as high or low density polyethylene, polycarbonate, acrylic, and/or PVC to allow natural or artificial light to penetrate through the external holding tank 37 and into the microorganism culture. In some embodiments, the external holding tank 37 may comprise a sterile aeration system to support the health of the microorganism culture. In some embodiments, the external holding tank 37 may comprise a cone-shaped base to ensure complete drainage of the microorganism culture when it is released onto a target field.

In some embodiments, the exterior holding tank 37 may comprise a cooling system such as a refrigerator to cool the microorganism culture during storage. The refrigerated exterior holding tank may be configured to receive the microorganism culture and/or microorganism slurry, maintain its sterility, and store it at any suitable temperature. For example, in some embodiments, the refrigerated exterior holding tank may maintain the microorganism culture and/or microorganism slurry at an optimal growth temperature where the temperature outside is warmer than the optimal growth temperature. In some embodiments, the refrigerated exterior holding tank may maintain the microorganism culture and/or microorganism slurry at a temperature sufficiently low to slow or stop growth of the microorganism. For example, the refrigerated exterior holding tank may maintain microorganism culture and/or microorganism slurry at 4° or a temperature just above freezing.

The microorganism culture may be distributed onto the target field through any suitable delivery method. For example, in some embodiments, an outlet of the external holding tank 37 may be coupled to any conventional irrigation system to facilitate delivery of the microorganism culture to the target field, such as sprinklers, drip irrigation systems, and/or sprayer systems. In some embodiments, the microorganism culture may be distributed through flooding the target field. In another embodiment, the microorganism culture may be distributed through aerial application onto the target field. In some embodiments, such as where the microorganism culture is delivered through sprayer or aerial application, additional water may be delivered to the target field to drive the microorganism into the soil.

Referring again to FIG. 5, the control system 516 automatically monitors and controls the soil enrichment system, for example according to user-defined parameters. The control system may comprise any suitable system for monitoring and controlling the soil enrichment system 500, such as a sensor and an automation controller. In one embodiment, the control system includes multiple sensors responsive to conditions of various other systems, and the automation controller receives signals from the sensors and adjusts the operation of the soil enrichment system accordingly.

For example, in some embodiments, the soil enrichment system 500 may comprise one or more sensors adapted to detect various aspects of the soil enrichment system 500 for monitoring the function of various components and/or monitoring conditions within the bioreactor and other systems. For example, in various embodiments, the one or more sensors may comprise a pH meter, a temperature sensor, a salinity sensor, a flow rate sensor, a nutrient concentration sensor, a turbidity sensor, a Photosynthetically Active Radiation (PAR) meter, a densitometer, a bioreactor capacity sensor, a liquid velocity sensor, a dissolved gas sensor, and/or a camera system.

In various embodiments, the sensors may detect aspects of the soil enrichment system 500 such as, but not limited to: a) growing conditions within the bioreactor, such as light level and/or temperature; b) microorganism cell titer/cell count in the water; c) pH of the water; d) salinity of the water; e) the presence of undesired microorganisms in the bioreactor, such as with a flow imaging device that creates images of the microorganism culture in the bioreactor; f) water level; g) level of nutrients in the neutralized irrigation water of the bioreactor; h) level of solids in the filtered irrigation water, the sterilized irrigation water, and/or the neutralized irrigation water; i) the level of undesired compound(s) in the neutralized irrigation water of the bioreactor; j) oxygen, ozone, and/or carbon dioxide content in the neutralized irrigation water of the bioreactor; k) level of nitrogenous compounds in the neutralized irrigation water of the bioreactor; l) clarity or opacity of the neutralized irrigation water of the bioreactor; m) level of desired compound(s) in the neutralized irrigation water of the bioreactor; n) flow-rate of neutralized irrigation water into the bioreactor; o) the presence of weed algae in the bioreactor; p) the presence of algal predators in the bioreactor; r) the presence of other contaminants in the bioreactor; and/or q) equipment status.

In various embodiments of the present technology, the sensors may be used to control operation of the system, such as by feedback regulation. Any sensor may generate one or more signals based on a condition sensed in the soil enrichment system and may send the one or more signals to one or more discrete controllers. The controllers may control the flow of materials into and/or out of the components of the soil enrichment system.

For example, in one embodiment, a sensor may detect a microorganism cell titer within the bioreactor and may send one or more signals corresponding to the microorganism cell titer to one or more flow controllers. The flow controllers may modify the flow of neutralized irrigation water and/or the nutrient solution into the bioreactor and/or the flow of microorganism culture out of the bioreactor based on the one or more signals. In a specific example, the flow controller may activate the flow of microorganism culture out of the bioreactor upon receiving a signal corresponding to a microorganism cell titer sufficiently high for harvest of the microorganism culture. The flow controller may then activate the flow of neutralized irrigation water and/or nutrient solution to fill the bioreactor for a subsequent inoculation.

In another example, a sensor comprising a pH monitor may detect the pH of the microorganism culture in the bioreactor. If the pH monitor detects an undesirably high (alkaline) pH, the pH monitor may send one or more signals to a carbon dioxide flow controller that controls the amount of, or rate at which, carbon dioxide is added to the bioreactor, causing the carbon dioxide flow controller to add carbon dioxide to the bioreactor to reduce the pH to a desirable level. In another embodiment, the pH monitor may send one or more signals to an acid or base titrating unit configured to control the amount of, or rate of, acid and/or base flowing into bioreactor to maintain a desirable pH.

In another example, a sensor comprising a water level monitor may detect the volume of the microorganism culture in the bioreactor. In some embodiments, the water level monitor may send one or more signals to a water flow controller to modify the amount of, or rate of, irrigation water flow into the soil enrichment system, neutralized irrigation water into the bioreactor, and/or microorganism culture out of the bioreactor in response to the state of the microorganism culture in the bioreactor.

The state of the microorganism culture in the bioreactor may correspond to the height (or level) of the bioreactor column reached by the microorganism culture and/or the health of the microorganism culture. A low column height may correspond to a low volume of microorganism culture, triggering activation of the water flow controller to allow neutralized irrigation water and/or nutrient solutions to enter the bioreactor. Likewise, column height corresponding to a desirable volume of microorganism culture in the bioreactor may trigger the water flow controller to stop the flow of neutralized irrigation water and/or nutrient solution into the bioreactor. Similarly, in another embodiment, a nutrient monitor may send one or more signals to a nutrient solution flow controller that may control the amount of, or rate at which, the nutrient solution is added to the bioreactor.

In some embodiments, a sensor comprising a water pressure monitor may detect the pressure of fluid traveling through the fluid conduits in the soil enrichment system. The water pressure monitor may send one or more signals to a water pressure regulator that may control the amount of, or rate of, irrigation water flowing into the soil enrichment system. In one embodiment, the water pressure regulator may be coupled to an outlet of the water source or to an inlet of the water storage tank.

In another embodiment, a sensor comprising an ozone monitor may send one or more signals to the ozone generator that controls the amount of, or rate at which, ozone is added to the water storage tank to sterilize the filtered irrigation water. In another embodiment, a sensor comprising a clarity monitor may send one or more signals to a water clarity controller that may control the efficiency of the solids filter.

Various embodiments of the control system 516 may comprise an automation controller, such as a programmable logic controller (PLC) system, to automatically control the operation of the soil enrichment system 500. In various embodiments, the automation controller includes a PLC system comprising a modular industrial computer control system configured to provide process control of the soil enrichment system 500. The PLC system may be communicatively linked to the components of the soil enrichment system including, but not limited to, the sensors, the bioreactor system 514, the sterilization system 17, the neutralization system 15, the blower 30, carbon dioxide source, lights, valves, cameras, pumps 18, and/or the automated cleaning system The PLC system may utilize the data received from the sensors and components to control the components of the soil enrichment system 500.

In various embodiments, the PLC system may provide at least one of an interface, either local, remote, or both, for a human operator or another controlling system to start, stop, or modify various parameters of the soil enrichment system 500. In some embodiments, the PLC system may be communicatively linked to other computers or PLC systems as a master, slave, or equal system The PLC system may be communicatively linked to an external computer to provide the operator with remote programming capabilities, additional user interface options, data storage, additional security, and/or additional computational power.

Various embodiments of the PLC system may employ any suitable type of cabled and/or wireless communication system such as light waves, radio waves, sound waves, infrared waves, ultraviolet waves, other such wavelengths/frequencies, media, and combinations thereof. In some embodiments, the PLC system may also employ an IP network (such as the Internet), GSM (global system for mobile communications) network, SMS (short message service) network, and combinations thereof.

Various embodiments of the PLC system may comprise one or more controllers. The one or more controllers may control the function of one or more components of the soil enrichment system. For example, the one or more controllers may control the flow of the nutrient solution into the bioreactor and/or control the delivery of carbon dioxide into the bioreactor from the carbon dioxide source. In some embodiments, the one or more controllers may facilitate a feedback loop. For example, irrigation water that has been improperly ozonated may be routed back through the water storage tank for further treatment with ozone. In another example, the one or more controllers may facilitate a feedback loop in which irrigation water that has been insufficiently filtered may be routed back into the first solids filter for further removal of solids.

In various embodiments, the PLC system may comprise a portable platform (or body or frame, not shown) onto which components of the PLC system may be mounted. Each of the components of the PLC system may be individually replaceable. Although the components may be indicated as single components, each of the components may be present in plurality independently of other components of the system Various operations of the PLC system may be performed under direct manual control and/or automatically. In some embodiments, the PLC system may comprise PLC code that may be customized to the PLC system and may be updated from time to time to change it functionality.

When used in conjunction with appropriate remote networking hardware and software, the PLC system may be controlled remotely from any location with Internet access. In some embodiments, multiple PLC systems in soil enrichment systems in different locations may be coordinated and controlled from a single location, allowing more capability than any single PLC system may provide.

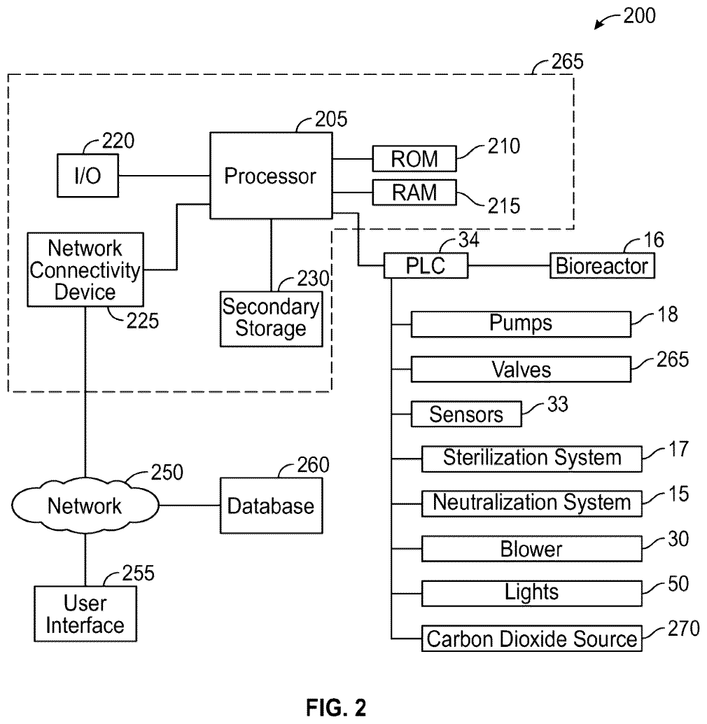

Referring to FIG. 2, an exemplary control system 200 suitable for implementing one or more of the present embodiments may include a computer system 265 communicatively linked to a PLC system 34. The PLC system 34 may be communicatively linked to the one or more sensors 33 and may provide measurements obtained by the one or more sensors 33 to a processor 205 and/or database 260 for remote monitoring, remote data access, and/or remote control of the soil enrichment system 100. The PLC system 34 may similarly be communicatively linked and configured to control pumps 18, valves, sterilization system 17, neutralization system 15, blower 30, lights 50, and/or carbon dioxide source 270.

The computer system 265 may include a processor 205 in communication with memory devices, such as read only memory (ROM) 210, random access memory (RAM) 215, and secondary storage 230. The processor 205 may also connect to one or more input/output (I/O) devices 220 and/or network connectivity devices 225.

The processor 205 may comprise logic circuitry to perform various functions in response to inputs. The processor 205 may execute instructions, codes, computer programs, scripts, and/or the like, which may be received or accessed from any suitable source. For example, the processor 205 may comprise any conventional digital processor that responds to and processes the basic instructions provided via a set of inputs. In one embodiment, the processor 205 may comprise a conventional central processing unit (CPU), such as a conventional microprocessor. The processor 205 may be implemented as one or more CPU chips. In one embodiment, the processor 205 may retrieve instructions from secondary storage 230, store them in RAM 215 for fast access, and execute the instructions for various tasks, such as retrieving and processing data from various sources.

In one embodiment, the processor 205 may be configured to process information and/or data received from the PLC system 34 that may be used to operate pumps, valves, the mineral solution pump 235, the carbon dioxide source 66, the automated cleaning system 70, the vitamin solution pump 240, and/or various sensors 33. The processor 205 may receive the information and/or data via a network connectivity device 225 configured to interface with a network 250, such as a cloud network, a local network, and/or a global network. For example, the PLC system 34 may first send the information and/or data it gathered from the sensors 33 to the processor 205 for processing. The processor 205 may then transmit the PLC system 34 information and/or data to other systems and/or other components of the soil enrichment system 500 via the network connectivity device 225.

In one embodiment, the processor 205 may transmit information and/or data to the network 250 using any suitable system or device configured to transmit information and/or data from a first source to a second source. For example, the processor 205 may be configured to transmit information and/or data wirelessly (WIFI, Bluetooth™, and/or the like) or non-wirelessly such as via a hardwire connection between the PLC system 34 and the network 250.

In one embodiment, the computer system 200 may be configured to interface with the database 260. The database 260 may comprise any suitable system configured to receive, store, and/or transmit information and/or data related to the computer system and its various components. The database 260 may be configured to transmit and/or receive information and/or data via the network 250. For example, information and/or data received or used by the PLC system 34 may be configured to be stored in the database 260.

In one embodiment, a user interface 255 may be configured to display information and/or data received by the PLC system 34. In another example, the user interface 255 may be configured to receive information and/or data from the database 260. The user interface 255 may transmit and/or receive information wirelessly (WIFI, Bluetooth™) and/or via a hardwire connection.

The I/O devices 220 may transfer information between the computer 265 and peripheral devices (not shown). For example, the I/O devices may include printers, video monitors such as liquid crystal displays (LCDs) and touch screen displays, keyboards, keypads, switches, dials, mice, track balls, voice recognizers, card readers, and the like. The computer system 265 may include interface systems to facilitate communications with the I/O devices 220, such as networking cards, graphics cards, USB ports, and the like.

The network connectivity device 225 may facilitate communications between the computer system 265 and one or more networks. The network connectivity devices may comprise any suitable network connectivity devices, such as network interface cards, hubs, switches, bridges, routers, gateways, repeaters, modems, modem banks, Ethernet cards, universal serial bus (USB) interface cards, serial interfaces, token ring cards, fiber distributed data interface (FDDI) cards, wireless local area network (WLAN) cards, and radio transceiver cards such as code division multiple access (CDMA) and/or global system for mobile communications (GSM) radio transceiver cards. The network connectivity devices 225 may also include one or more transmitters and receivers for wirelessly or otherwise transmitting and receiving signals.

The network connectivity devices 225 may enable the processor 205 to communicate with the network 250, such as an Internet or one or more intranets. By operating in conjunction with the network 250, the computer system 265 may receive information from the network 250 and/or output information to the network 250 in the course of performing the monitoring processes and operation functions of the soil enrichment system.

Such information, which may include a sequence of instructions to be executed using the processor 205, may be received from and outputted to the network 250 via a transmission medium. The transmission medium may comprise any appropriate medium for communicating information, such as electrical signals, optical signals, wireless connection, and/or RF communications. In one embodiment, information is communicated in the form of a computer data baseband signal or signal embodied in a carrier wave.

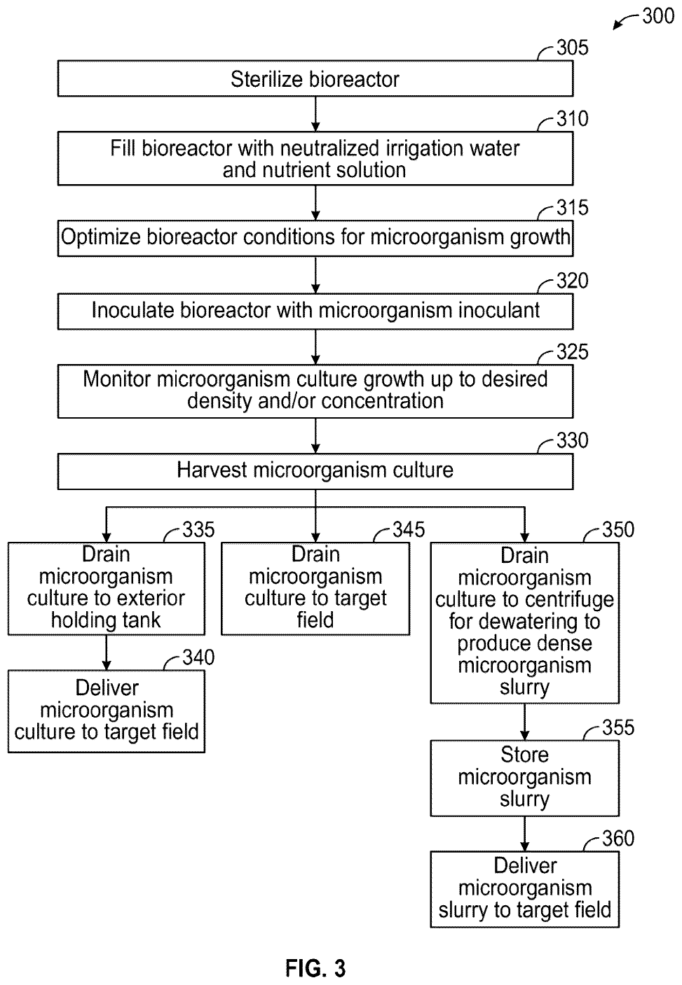

Referring to FIGS. 1 and 3, an exemplary method of operating the soil enrichment system 100 (300) may comprise initially sterilizing one or more bioreactors 16 prior to inoculation with the microorganism inoculant (305). For example, the one or more bioreactors 16 may be sterilized with a bleach or alcohol solution.

After sterilization of the bioreactors 16 and/or the various other elements of the soil enrichment system 500, the bioreactors 16 may be filled with neutralized irrigation water and nutrient solution (310). For example, multiple pumps 18, such as peristaltic pumps, may propel irrigation water from the water source 5 through fluid conduits 10. The irrigation water may be filtered through the first solids filter 19 coupled to the water source 5 and stored in the water storage tank 12. The water storage tank 12 may be coupled to the sterilization system 17, such as the ozone generator. The ozone generator may be coupled to an oxygen concentrator (not shown). The ozone generator may be configured to generate ozone and deliver the ozone to the filtered irrigation water in the water storage tank 12 to form sterilized irrigation water.

The sterilized irrigation water may exit the water storage tank 12 and be propelled through another pump 18 to the second solids filter 60 positioned immediately downstream to the water storage tank 12 and the pump 18. The second solids filter 60 may be configured to remove solids in the sterilized irrigation water.

After filtration, the sterilized irrigation water may pass through the neutralization system 15. The neutralization system 15 may comprise the carbon filter and/or the UV light system. The neutralization system 15 may be positioned immediately downstream from the second solids filter 60 and configured to remove ozone from the filtered sterilized irrigation water to form neutralized irrigation water.

A first nutrient solution container 20 comprising the nutrient solution may be positioned immediately downstream of the neutralization system 15. Another pump 18 may be configured to conduct the nutrient solution in the first nutrient solution container 20 into the fluid conduit 10 immediately downstream of the neutralization system 15. As discussed above, the first nutrient solution container 20 may be positioned downstream of the neutralization system 15 to avoid degradation of the nutrient solution. The nutrient solution may, in some embodiments, need refrigeration. For example, the vitamin solution discussed above may be refrigerated to preserve the vitamins. The first nutrient solution container 20 may be kept within a refrigerator 20.

A second nutrient solution container 62 comprising a second nutrient solution may also be positioned immediately downstream of the neutralization system 15. The second nutrient solution may not need refrigeration, such as the mineral solution discussed above. Another pump 18 may be configured to conduct the second nutrient solution in the second nutrient solution container 62 into the fluid conduit 10 immediately downstream of the neutralization system 15.

The neutralized irrigation water containing nutrient solution may be conducted into any one or more of the bioreactors 16 until it reaches a preselected fill level 40. The one or more bioreactors growth conditions may then be optimized for microorganism growth including temperature, pH, and/or light intensity (315).

The neutralized irrigation water and nutrient solution in the one or more bioreactors may be inoculated with the microorganism inoculate and grown to form a microorganism culture (320). The one or more bioreactors 16 may be inoculated with the microorganism inoculant by any suitable method, such as manual inoculation through a port 35 in the bioreactor 16.

A light source 45/50 may be configured to project light onto and/or into each of the one or more bioreactors 16. In some embodiments, the light source 45/50 may comprise LED lights in any suitable configuration to provide light to the microorganism culture. For example, in one embodiment, a first light source 45 may be positioned within the one or more bioreactors 16. In another embodiment, the first light source 45 may overlay an exterior surface of the one or more bioreactors 16. In another embodiment, a second light source 50 may be outside of and adjacent to an exterior surface of the one or more bioreactors 16.

In various embodiments, the contents of the one or more bioreactors 16 may be mixed by use of air bubbles produced by the blower 30. The blower 30 may conduct air to an air diffuser in the base of the one or more bioreactors 16 (not shown). One or more sensors 33 in the one or more bioreactors 16 may measure various parameters such as pH, temperature, cell density, water mixing velocity, dissolved gasses and proteins as discussed above. This exemplary embodiment of the soil enrichment system 100 may be suitable for low, medium, and high volume irrigation applications to a target field 55 and/or flowing to the exterior holding tank 37. In some embodiments, the exterior holding tank 37 may sit on a trailer for portability (trailer not shown).

The growth of the microorganism culture may be monitored, such as through the sensors 33 and the PLC system 34, and growth conditions within the one or more bioreactors 16 may be adjusted as needed. The microorganism culture may be allowed to grow until it reaches a density appropriate for agricultural use, such as at least 1 million cells per milliliter (325).

Once the microorganism culture reaches a desired density, the microorganism culture may be harvested (330). The drain valve of the one or more bioreactors 16 may be opened and the drain pump may be activated to deliver the microorganism culture onto the target field (345) and/or into an exterior holding tank (335). In some embodiments, the drain valve may comprise an actuated ball valve controlled by the PLC system 34. Microorganism culture delivered into the exterior holding tank may subsequently be drained onto the target field (340). In some embodiments, the microorganism culture may be delivered into the centrifuge for dewatering to produce the dense microorganism slurry (350). The dense microorganism slurry may be stored (355) and ultimately delivered to the target field (360).

The size or operating capacity of each component of the soil enrichment system may be varied as needed. In an exemplary embodiment, the soil enrichment system comprising a total bioreactor capacity of about 500 gallons of microorganism culture may support treatment of about 500 acres of land (i.e., target field) and may generally comprise the following minimum operating capacities for the indicated components: a) ozone source—about 12 g/hr; (O₃) at about 10 standard cubic feet per hour (scfh) O₂; b) a first solids filter—about 55 gallon/minute maximum flow rate with a minimum surface area of about 2 ft²; c) a second solids filter—about 25 gallon/minute minimum flow rate with a minimum surface area of about 1 ft²; d) carbon filter—about 2 ft³ minimum or UV filter of about 25 gallons per minute (GPM) minimum; e) water pump—about 25 gallon/minute minimum flow rate; f) blower—about 50 actual cubic feet per minute (ACFM) flow rate at about 14 pounds per square inch absolute (PSIA) minimum; g) microorganism culture—about 1.0×10⁶ cells/milliliter minimum; h) liquid carbon dioxide source—about 80 liter/week.

In the foregoing description, the technology has been described with reference to specific exemplary embodiments. Various modifications and changes may be made, however, without departing from the scope of the present technology as set forth. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present technology and are accordingly not limited to the specific configuration recited in the specific examples.

Benefits, other advantages, and solutions to problems have been described above with regard to particular embodiments. Any benefit, advantage, solution to problems, or any element that may cause any particular benefit, advantage or solution to occur or to become more pronounced, however, is not to be construed as a critical, required, or essential feature or component.

The terms “comprises,” “comprising,” or any variation thereof, are intended to reference a nonexclusive inclusion, such that a process, method, article, composition, system, or apparatus that comprises a list of elements does not include only those elements recited, but may also include other elements not expressly listed or inherent to such process, method, article, composition, system, or apparatus. Other combinations and/or modifications of the above-described structures, arrangements, applications, proportions, elements, materials, or components used in the practice of the present technology, in addition to those not specifically recited, may be varied or otherwise particularly adapted to specific environments, manufacturing specifications, design parameters, or other operating requirements without departing from the general principles of the same.

The present technology has been described above with reference to an exemplary embodiment. However, changes and modifications may be made to the exemplary embodiment without departing from the scope of the present technology. These and other changes or modifications are intended to be included within the scope of the present technology.

1-35. (canceled)

36. A soil enrichment system comprising:
an inlet,
a fluid conduit,
a first solids filter,
a water storage tank,
a sterilization system,
an ozone generator,
a gas diffuser,
a neutralization system,
a bioreactor system, and
a control system;
wherein the inlet is coupled to the fluid conduit;
wherein the first solids filter is coupled to the fluid conduit;
wherein the water storage tank is coupled to the fluid conduit downstream of the first solids filter and the inlet;
wherein the sterilization system is coupled to the water storage tank;
wherein the sterilization system comprises the ozone generator;
wherein the sterilization system is configured to deliver ozone gas from the ozone generator to irrigation water in the water storage tank to form sterilized irrigation water;
wherein the control system is configured to apply a concentration of the ozone gas to irrigation water in a range of approximately 0.2 parts per million (ppm) to approximately 0.5 ppm;
wherein the water storage tank comprises the gas diffuser;
wherein the gas diffuser is configured to distribute the ozone gas to the irrigation water;
wherein the neutralization system is positioned downstream from the water storage tank; wherein the neutralization system is configured to remove ozone from the sterilized irrigation water to form neutralized irrigation water;
wherein the neutralization system is configured to prevent chemicals used to produce the sterilized irrigation water from killing and/or impeding growth of microbes grown in the bioreactor system;
wherein the neutralization system comprises an ultraviolet (UV) light system;
wherein the UV light system is configured to expose the sterilized irrigation water to UV radiation to degrade the ozone;
wherein the UV light system is configured as a flow-through system in which the sterilized irrigation water is deozonated as it passes through the UV light system;
wherein the bioreactor system is coupled to the neutralization system;
wherein the bioreactor system comprises a microalgae growth chamber configured to receive a microalgae inoculant;
wherein the control system is configured to activate a flow of microalgae culture out of the bioreactor system upon receiving a signal corresponding to a microalgae cell titer sufficiently high for harvest of the microalgae culture; and
wherein the control system is configured to activate a flow of the neutralized irrigation water to fill the bioreactor system for a subsequent inoculation after the harvest of the microalgae culture.

37. The soil enrichment system of claim 36,
wherein the harvest of the microalgae culture includes live microalgae.

38. The soil enrichment system of claim 37,
wherein the sterilization system further comprises a chlorine generator.

39. The soil enrichment system of claim 38,
wherein the chlorine generator comprises a chlorine generator cell that produces hypochlorous acid from salt.

40. The soil enrichment system of claim 39,
further including a dosing pump;
wherein the control system is configured to inject a dose of the hypochlorous acid into the water storage tank.

41. The soil enrichment system of claim 40,
wherein the UV light system is configured to expose the sterilized irrigation water to the UV radiation to remove chlorine.

42. The soil enrichment system of claim 41,
wherein the water storage tank comprises a cone-shaped bottom.

43. The soil enrichment system of claim 42,
where in the water storage tank comprises a light blocking material configured to reduce exposure of the irrigation water to light to impeded growth of unwanted microbes.

44. The soil enrichment system of claim 37,
wherein the soil enrichment system is a portable soil enrichment system.

45. The soil enrichment system of claim 44,
wherein the portable soil enrichment system is configured to deliver the live microalgae directly onto a target field.

46. The soil enrichment system of claim 45,
wherein the portable soil enrichment system is disposed within a portable housing.

47. The soil enrichment system of claim 46,
wherein the portable housing includes environmental controls.

48. The soil enrichment system of claim 47,
wherein the environmental controls are configured to regulate an ambient environment within the portable housing.

49. The soil enrichment system of claim 48,
wherein the environmental controls include air conditioning.

50. The soil enrichment system of claim 49,
wherein the environmental controls include humidity control systems.

51. The soil enrichment system of claim 50,
wherein the portable housing includes doors configured to provide an airtight seal to inhibit contamination of the bioreactor system.

52. The soil enrichment system of claim 50,
wherein the portable housing is configured to couple to a water source.

53. The soil enrichment system of claim 52,
wherein the portable housing includes on opening for a fluid conduit carrying the microalgae culture.

54. The soil enrichment system of claim 48,
wherein the portable housing includes a shipping container.

55. The soil enrichment system of claim 54,
wherein the shipping container is configured to be lifted onto a truck, trailer, and/or train by a forklift or a crane.
